(19) United States
(12) Patent Application Publication
(10) Pub. No.: US 2004/0248275 A1
(43) Pub. Date: Dec. 9, 2004

(54) Engineered bacterial aggregates

(76) Inventors: Peter Gilbert, Cheshire (GB); Alexander H. Rickard, Washington, DC (US)

Correspondence Address: S.C. JOHNSON & SON, INC., 1525 HOWE STREET, RACINE, WI, 53403-2236, US

(21) Appl. No.: 10/811,651

(22) Filed: Mar. 29, 2004

Related U.S. Application Data
(60) Provisional application No. 60/459,471, filed on Apr. 1, 2003.

(51) Int. Cl. C12N 1/20

(52) U.S. Cl. 435/252.1

(57) ABSTRACT

A method of creating a bacterial aggregate comprising the step of combining planktonic bacterial cells with an effective amount of lectin, wherein the amount of lectin is effective to bind the bacterial cells together in an aggregate, is disclosed.

### CROSS-REFERENCE TO RELATED APPLICATION

This application claims priority to U.S. provisional application 60/459,471, filed Apr. 1, 2003, incorporated herein by reference.

### STATEMENT REGARDING FEDERALLY SPONSORED RESEARCH OR DEVELOPMENT

### BACKGROUND OF THE INVENTION

In the natural environment bacteria seldom exist as single free-floating cells. Rather, the majority of bacteria exist as part of aggregated communities such as biofilms or floccules. Polymeric materials such as polysaccharides and glycoproteins often, but not necessarily, envelop these. Biofilms are highly resistant to antimicrobial formulations. The simplest type of biofilm is a bacterial aggregate.

These aggregates possess many of the characteristics of classic surface-bound biofilms, including resistance to biocides and antibiotics. A variety of reasons have been suggested. These mainly focus upon the close proximity of cells and the exclusion properties of the polymeric matrix. The latter, however, has been shown to possess a diffusivity that is similar to water and will not retard biocide penetration unless the two interact chemically or ionically. The likely resistance mechanisms therefore center upon the close proximity of cells that would provide a tortuous path for diffusion and might retard penetration, together with possible compartmentalization of the community through an arrangement of hydrophobic and hydrophilic mosaic compartments.

The ability to construct simple and reproducible bacterial aggregates has multiple applications. In the laboratory, the aggregates would guide the formulation of biocidal products intended for application in hygienic situations. In the field, such ability would have application towards biotransformation and bioremedioration.

### BRIEF SUMMARY OF THE INVENTION

In one embodiment, the present invention is a method of creating a bacterial aggregate comprising the step of combining planktonic bacterial cells with an effective amount of lectin, preferably concanavalin A, wherein the amount of lectin is effective to bind the bacterial cells together in an aggregate. The cells may be homogenous or heterogeneous. The invention is also an aggregate created by this method.

In another embodiment, the invention additionally comprises the step of coating the bacterial aggregate with a second mixture of bacteria and lectin, whereby a lamellar aggregate is constructed. One may also wish to use third, or any number, of mixtures. The invention is also an aggregate created by this method.

In another embodiment, the present invention is a method of evaluating the efficacy of a biocide comprising the step of exposing the bacterial aggregates of the invention to the biocide and evaluating the viability of the bacterial cells within the aggregate.

In another embodiment, the present invention is a method of creating a microbial aggregate comprising the step of combining microbes with an effective amount of lectin, wherein the amount of lectin is effective to bind the microbes together in an aggregate. Preferably, the microbes comprise at least one member from the group consisting of bacteria, yeast and fungi. The invention is also an aggregate created by this method and a method of evaluating the efficacy of a biocide comprising the step of exposing the aggregate to the biocide and evaluating the viability of organisms within the aggregate.

### BRIEF DESCRIPTION OF THE SEVERAL VIEWS OF THE DRAWINGS

FIG. 1 is a set of light micrographs showing the aggregation of free-floating Pseudomonas sp. 2881 by the addition of ConA. FIG. 1A is a free floating Pseudomonas sp. 2881 without ConA and FIG. 1B Pseudomonas sp. 2881 aggregated with 0.1 mg/ml ConA.

FIG. 2 is a diagram showing the parabolic relationship between ConA concentration/cell density ratio and the size of the aggregate formed.

FIG. 3 is a diagram showing the different approaches used to construct mosaic or multi-species aggregates using ConA lectin. FIG. 3A are simple mosaics formed by mixing aggregates of one organisms created under a lectin deficiency with those formed under lectin excess. FIG. 3B are free-floating bacteria added to aggregates formed under a lectin-excess in order to create a lamellar structure. FIG. 3C is unordered aggregates constructed by adding lectin to a mixture of free-floating bacteria.

FIG. 4 is a set of light micrographs digitally combined with florescence micrographs of multi-species or mosaic aggregates. To easily visualize the spatial positions of species within the aggregates, one species of bacteria was engineered to express Green Fluorescent Protein (GFP). FIG. 4A: Unordered mixture of Pseudomonas sp. 2881 expressing GFP and A. hydrophila. FIG. 4B: Ordered mixture of A. hydrophila expressing GFP (core) and C. aquaticum (shell) FIG. 4C: Ordered mixture of A. hydrophila (core) and Pseudomonas sp. 2881 expressing GFP (shell). Bar represents 10 μm.

FIG. 5 is a graph showing the changes in susceptibility of populations associated with the aggregation of Pseudomonas sp. 2881. 1 mM QUAT used. Standard error within 5%.

FIG. 6 is an image of the precision syringe driver prepared for use with attached bijou, syringe and air filter.

FIG. 7 is a graph of sample data showing the influence of gradual application of 1 mM C14 QUAT to Pseudomonas sp. 2881. Duration of QUAT addition to achieve 1 mM is shown. Control represents no treatment.

FIG. 8 is a graph of sample data showing a comparison of the time survival kinetics of single cell suspensions of Pseudomonas sp. 2881 and C. aquaticum (A) and a mixed suspension of Pseudomonas sp. 2881 and C. aquaticum (B) following treatment with 0.05 mM C12 QUAT: FIG. 8A: —♦—: Suspension of non-aggregated Psuedomonas sp. 2881. —▪—: Suspension of non-aggregated C. aquaticum. Standard error within 5%. FIG. 8B: Suspension of non-aggregated Pseudomonas sp. 2881 cells (-x-) mixed with a suspension of C. aquaticum cells (-x-). Standard error within 5%.

### DESCRIPTION OF THE INVENTION

One object of the present invention is to construct microbial aggregates of known size and with defined spatial organization of the contained species. Microbial species to be selected would represent extremes of susceptibility and binding affinity for the model biocides (e.g., quaternary ammonium compounds [QACs or QUATs]). Susceptibility of the organisms towards various biocides could then be assessed for different aggregate sizes and for different community architectures.

In one embodiment of the present invention, artificial microbial aggregates were constructed using planktonic cells bound to one-another by lectins. Lectins are non-enzymatic sugar-binding proteins or glycoproteins of non-immune origin (Goldstein, et al., Nature 285:86, 1980) which react with specified terminal sugar residues. Lectins have been used to agglutinate cells and are useful probes for the study of carbohydrates displayed on cell surfaces.

We have selected the lectin Concanavalin A (ConA) as the most preferred (Sigma, Poole, Dorset, UK). Other lectins tested for their potential in the present invention were Wheat Germ Lectin (WGA), lectin from Limulus polyphemus and Peanut Agglutinin (PNA). All showed some suitability for the construction of aggregates. However, ConA was the only lectin that strongly reacted with, and aggregated, over 15 taxonomically distinct species of bacteria. ConA was subsequently deemed the most preferable for use. However, any lectin is suitable for the invention as long as the lectin is capable of agglutinating the desired species.

Lectins may be deployed to engineer aggregates of different bacterial species. In one embodiment of the present invention, the sequence of lectin addition to the component organisms enables clonal mosaic structures to be rapidly constructed. By “clonal mosaic” we mean structure where each mosaic component is comprised of a cluster (aggregate) of an individual clone. In one example the mosaic would comprise multiple patches where each patch was 20-100 aggregated bacteria of the same heritage. Alternatively, the formed constructs might be multi-lamellar spheres or homogeneous mixtures of the partner organisms. The constructs may also be heterogeneous, i.e. a mix of different organisms.

In preferred versions of the method of the present invention, one would create a bacterial aggregate as follows:

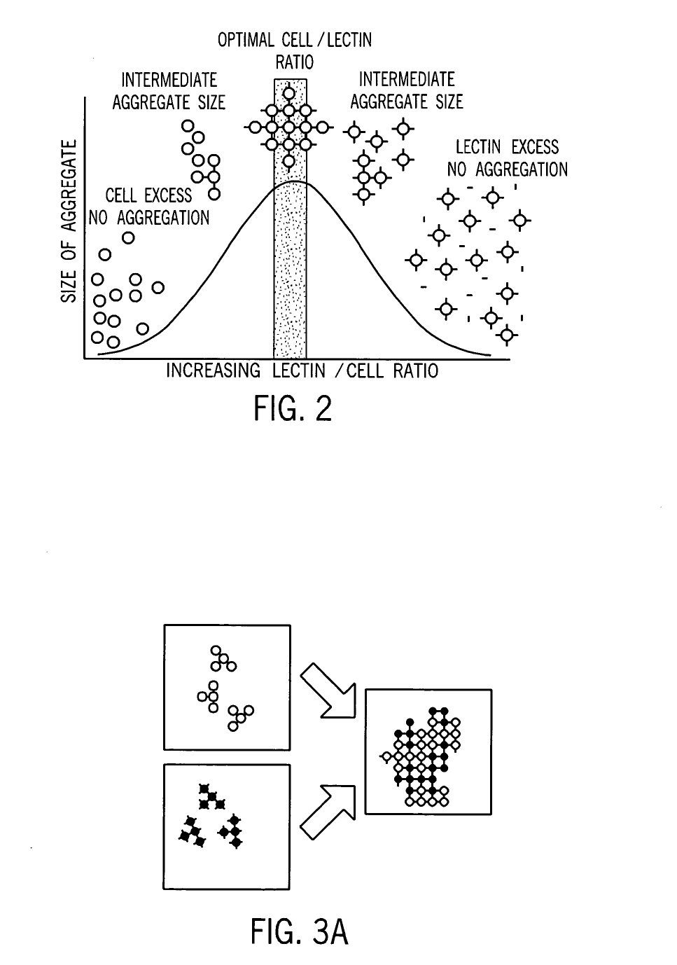

(i) In order to construct simple aggregates of single species of microorganisms (homogeneous) the following method is preferred: The microorganisms will have been found suitable with the lectin ConA. Of these, the Pseudomonas and Escherichia isolates are particularly appropriate for biocide testing. To a suspension of cells (1×10⁷-5×10⁸ cells/mL) one would add an equal volume of various concentrations of the appropriate lectin [see Table 2] (1×10⁻⁵-1 mg/mL). Examine under phase contrast microscopy in order to estimate the size of aggregates generated. The relationship between aggregate size and lectin concentration will be parabolic (FIG. 2). Select the appropriate size of aggregate from either the high (XS) or low (LIM) lectin concentration range.

(ii) In order to construct simple multispecies aggregates (heterogeneous) the following method is preferred: To a suspension (total cell density of 1×10⁷-5×10⁸ cells/mL) comprising the desired ration of chosen species (must all show affinity for the chosen lectin) one would add an equal volume of various concentrations of the appropriate lectin [see Table 2] (1×10⁻⁵-1 mg/mL). One would examine under phase contrast microscopy in order to estimate the size of aggregates generated. The relationship between aggregate size and lectin concentration will be parabolic (FIG. 2). Select the appropriate size of aggregate from either the high (XS) or low (LIM) lectin concentration range.

(iii) In order to construct binary mosaics of different species, one would then mix to the desired volume ratios homogeneous aggregates (i, above) of appropriate size where the major component was XS and the other LIM. Heterogenous and homogeneous aggregates may be combined or substituted, as appropriate, in the construction of designer mosaics.

It has been demonstrated that the nature of these aggregates affects susceptibility of the enveloped cells to biocidal formulations and that this change in susceptibility relates to a retardation of biocide equilibration within the aggregate (biofilm) core. The addition of permeabilisers to biocide formulations will enhance biocidal outcomes only if the equilibrium is achieved within 30 seconds. The biofilm constructs described here will serve as a convenient, reproducible laboratory model by which the efficacy of formulations can be benchmarked and developed.

Whilst not described by the presented data, lectins will also aggregate eukaryotic cells such as yeasts and fungi. One might wish to construct a biofilm of mixed species or single species selected from the group consisting of bacteria, yeast and fungi.

In many bioremediation systems floccules of bacteria develop over time that represent structured, ordered consortia of bacteria that collectively conduct the necessary biotransformations/detoxifications. These take time to establish themselves and can easily be lost to the environment if it is perturbed by an external stress. The invention provides a means by which such constructs might be engineered in the laboratory/factory and transported for use in the field.

Published work has demonstrated that even obligate anaerobic organisms such as Bacteriodes sp. and Fusobacteria can survive within an aggregate of strongly aerobic organisms. Normally such bacteria would be killed by contact with air. Lectin-mediated constructs would therefore provide a means whereby such organisms could be formulated into products, stored and transported.

Multi-lamellar spherical aggregates can be engineered such that they optimize the desired physiological activities of the partner organisms for use in commercial processes.

In another method of the present invention, one would form a multilamellar spherical aggregate in the following manner: In order to construct lamellar aggregates, aggregates of defined size should be prepared for the ‘core’ under XS (see i or ii above). Cores, either homogeneous or heterogeneous, are harvested by centrifugation (3000 g 10 minutes) and resuspended in the original volume of phosphate buffered saline (pH 7.1). Mix equal parts of the harvested core aggregates and a washed suspension of the ‘coating organism’ (1×10⁷-5×10⁸ cells/mL).

In another version of the present invention, one would test a biocide with the aggregates described above in the following manner: Volumes (1-10 mL) of customized aggregate suspensions, formed as described earlier, are held in suitable containers (microtitre plate well, mini-centrifuge tubes, pyrex glass test-tubes) to which are added appropriate concentrations of the test biocide (volume 5-25% of suspension volume). After a chosen contact times have elapsed (1-30 minutes) samples are removed to a neutralizer solution appropriate to the chosen biocide containing the antagonistic sugar (50 mM) for the chosen lectin (See Table 1). The aggregates disperse into single cell suspension that upon which simple plate count estimates of the viable surviving cell number may be conducted. Control experiments are conducted on disaggregated populations created by resuspending the customized aggregates in the antagonistic sugar (50 mM) prior to the addition of biocide.

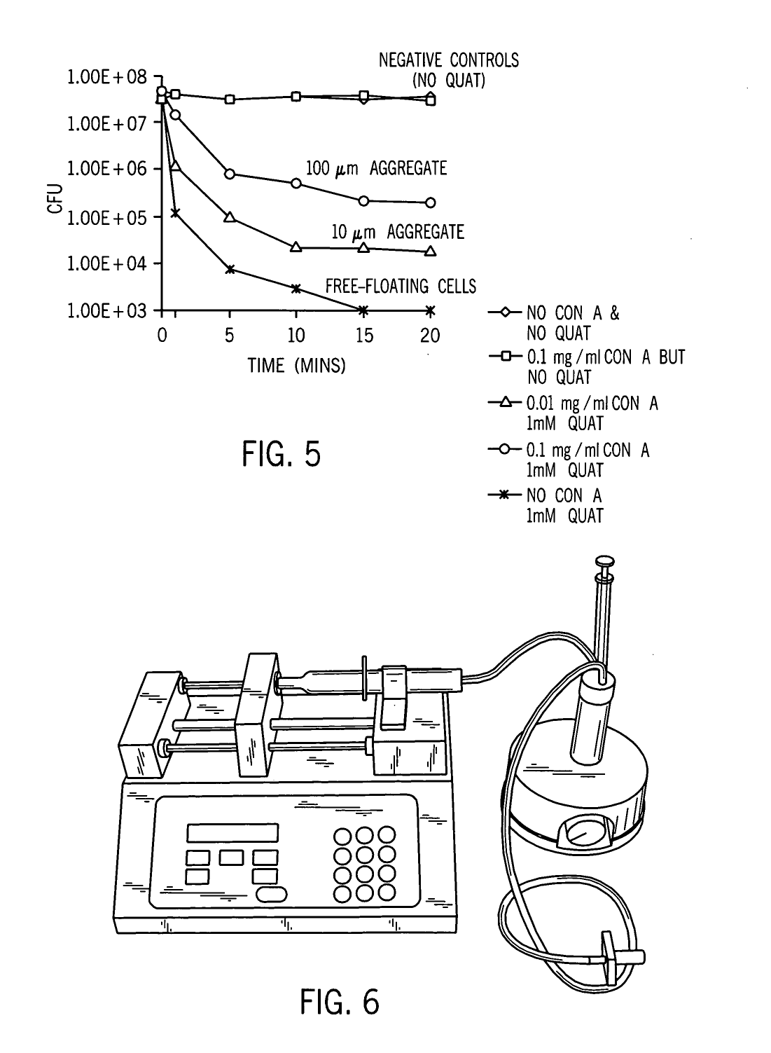

The effect of aggregation and of aggregate size is shown in FIG. 5 for a QAC (C=14) biocide. Reductions in killing occur in proportion to the size of the aggregate and the spatial geometry of the aggregated species (FIG. 8 and Tables 6, 7 and 8). The utility of biocide formulations against biofilm communities will be indicated by the lack of difference between the results of disinfection experiments performed on aggregated and disaggregated suspensions.

Whilst the lectin-mediated constructs here can be disassembled by the addition of an antagonizing sugar or by the production of certain extracellular protease enzymes by the component bacteria, it is envisaged that the aggregates might be stabilized once formed by various stable polymers, viz. aggregates formed within solutions of alginate might be filtered to remove excess alginate and ‘fixed’ by addition of calcium salts. Alternatively, the aggregates might be deployed as the catalytic shell in polymer microencapsulation.

### EXAMPLES

A. Reactivity of Different Lectins to Panel of Different Species of Bacteria.

Assay approach: Six lectins were chosen to discover their suitability for the aggregation of different species of bacteria. These were Concanavalin A, Lentil lectin, Pseudomonas aeruginosa lectin, peanut lectin, Limulus polyphemus lectin and wheat germ lectin. The specificities and antagonistic sugars for these lectins are shown in Table 1 and Table 2, below.

TABLE 1
Specificity of lectins used to aggregate
different species of bacteria.

| Lectin | Antagonistic Sugar |
|---|---|
| Concanavalin A | Mannose and glucose |
| Lentil lectin | Mannose and glucose |
| Pseudomonas aeruginosa lectin | Galactose and fucose |
| Peanut lectin | Galactose |
| Limulus polyphemus lectin | N-acetyl-D-galactosamine |
| Wheat germ lectin | N-acetyl-D-glucosamine |

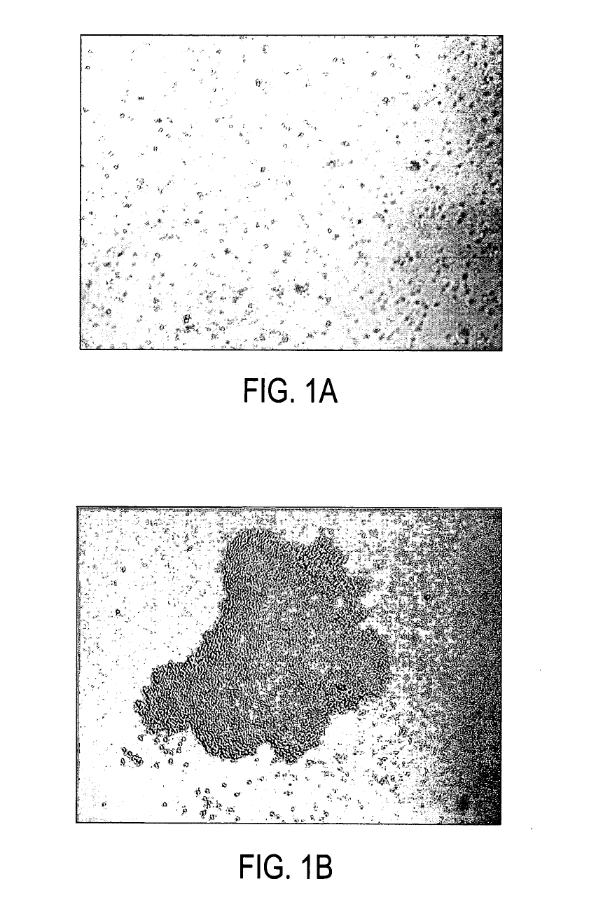

The ability of the lectins to aggregate bacterial cells was studied by mixing cells at an O.D. of 0.5 at 650 nm with different lectins at different concentrations. This was carried out in 96 well microtitre wells (example shown in FIG. 1). The extent of aggregation was determined by a semi-quantitative approach. Where the addition of lectins did not cause the aggregation of cells, a score of “0” was assigned. Lectins that caused cellular-aggregation to give small flocs in a turbid suspension were given a score of “+”. A score of “++” was given to lectin-cell aggregate mixes that resulted in large aggregates seen by eye in a turbid suspension. Lectins that caused cellular aggregation to give easily visible aggregates in a clear solution were given a score of “+++”. Results are shown in Table 2.

TABLE 2
The influence of different concentrations of lectins on the aggregation of different
species of bacteria. Concentrations are mg/mL

| | | | | | | | | | | | | | Limulus | | | | | |
|---|---|---|---|---|---|---|---|---|---|---|---|---|---|---|---|---|---|---|
| | Concanavalin | | | | | | P. aeruginosa | | | | | | polyphemus | | | Wheat | | |
| | A | | | Lentil lectin | | | lectin | | | Peanut lectin | | | lectin | | | Germ Lectin | | |
| | 1 × | 1 × | 1 × | 1 × | 1 × | 1 × | 1 × | 1 × | 1 × | 1 × | 1 × | 1 × | 1 × | 1 × | 1 × | 1 × | 1 × | 1 × |
| Strain | 10⁻¹ | 10⁻² | 10⁻³ | 10⁻¹ | 10⁻² | 10⁻³ | 10⁻¹ | 10⁻² | 10⁻³ | 10⁻¹ | 10⁻² | 10⁻³ | 10⁻¹ | 10⁻² | 10⁻³ | 10⁻¹ | 10⁻² | 10⁻³ |
| K. pneumoniae | +++ | + | + | ++ | + | + | + | +++ | ++ | + | ++ | + | 0 | 0 | 0 | ++ | + | 0 |
| K. oxytoca | ++ | + | 0 | ++ | + | 0 | ++ | + | + | ++ | + | + | 0 | 0 | 0 | + | + | 0 |
| P. putida | +++ | ++ | + | +++ | ++ | + | +++ | + | + | +++ | + | + | ++ | + | + | 0 | 0 | 0 |
| S. plymuthica | + | + | + | + | + | + | + | + | + | + | + | + | + | 0 | 0 | 0 | 0 | 0 |
| A. hydrophila | +++ | ++ | ++ | +++ | ++ | ++ | +++ | ++ | ++ | ++ | ++ | ++ | 0 | 0 | + | 0 | 0 | 0 |
| S. paucimobilis | + | ++ | 0 | + | 0 | 0 | ++ | + | + | ++ | + | + | + | ++ | 0 | 0 | 0 | 0 |
| C. freundii | +++ | + | + | +++ | + | + | 0 | 0 | 0 | 0 | 0 | 0 | 0 | 0 | 0 | 0 | 0 | + |
| E. brevis | + | + | 0 | + | 0 | 0 | + | + | 0 | ++ | + | + | + | 0 | 0 | 0 | 0 | 0 |
| E. asburiae | + | + | 0 | 0 | 0 | 0 | ++ | ++ | + | ++ | ++ | + | 0 | 0 | 0 | 0 | 0 | 0 |
| S. haemolyticus | +++ | ++ | + | +++ | + | + | +++ | + | + | +++ | + | + | 0 | 0 | 0 | +++ | ++ | + |
| E. faecalis | 0 | + | ++ | + | + | ++ | + | ++ | + | + | ++ | 0 | 0 | 0 | 0 | 0 | 0 | 0 |
| C. aquaticum | ++ | ++ | 0 | ++ | + | 0 | 0 | 0 | 0 | 0 | 0 | 0 | 0 | 0 | 0 | ++ | + | 0 |
| Pseudomonas | +++ | ++ | ++ | +++ | ++ | + | + | 0 | 0 | + | 0 | 0 | ++ | + | + | 0 | 0 | 0 |
| sp. 2881 | | | | | | | | | | | | | | | | | | |
| E. coli S17-1 | +++ | +++ | + | +++ | ++ | + | ++ | + | + | +++ | ++ | + | ++ | ++ | 0 | 0 | 0 | 0 |
| (K12) | | | | | | | | | | | | | | | | | | |
| E. coli C600 | +++ | +++ | + | +++ | ++ | + | ++ | + | + | +++ | ++ | + | +++ | + | 0 | 0 | 0 | 0 |
| (K12) | | | | | | | | | | | | | | | | | | |

B. Construction of Single Species Microbial Aggregates

Aggregates of different size were constructed by titrating cona against suspensions of bacteria and observing the formation of aggregates. These were visible by eye as well as by optical microscope (FIG. 1).

Aggregate size was measured. A parabolic relationship between the ratio of ConA and bacterial density was observed (Table 3). It is likely that this can be explained in terms of regions of lectin and cell excess, where no aggregation would occur and ranges where the cells or lectin were in slight excess (FIG. 2). This hypothesis reflects that established for antibody:antigen precipitation reactions.

Within an optimal range of 1×10⁻³ cfu cells and 0.6 mg/ml ConA, bacterial aggregates of 10 to >100 μm diameter could be reproducibly formed with a lectin excess that enabled further bacteria to be sequestered to the surface of the aggregate. The aggregates could be readily disassociated by the addition of approximately 50 mM maltose, glucose or sucrose to ConA, followed by 30s of gentle pipetting. This is ideal for the accurate enumeration of viable bacteria based upon CFU measurement on nutrient agar.

TABLE 3
The amount of ConA required to aggregate single
“free-floating” cell suspensions of
Pseudomonas sp. Strain 2881 to a defined size.
Size of aggregate within 5.0% S.E.

| | Amount of ConA | Size of aggregate |
|---|---|---|
| Strain | (mg/ml) | (μm) |
| Pseudomonas sp. | 0.001 | No aggregation |
| Strain 2881 | 0.01 | 11.2 |
| | 0.05 | 53.4 |
| | 0.1 | 106.7 |
| | 0.2 | 91.2 |
| | 0.4 | 73.1 |
| | 0.6 | 55.4 |
| | 0.8 | No aggregation |
| | 1.0 | No aggregation |

C. Construction of Mosaic Microbial Aggregates

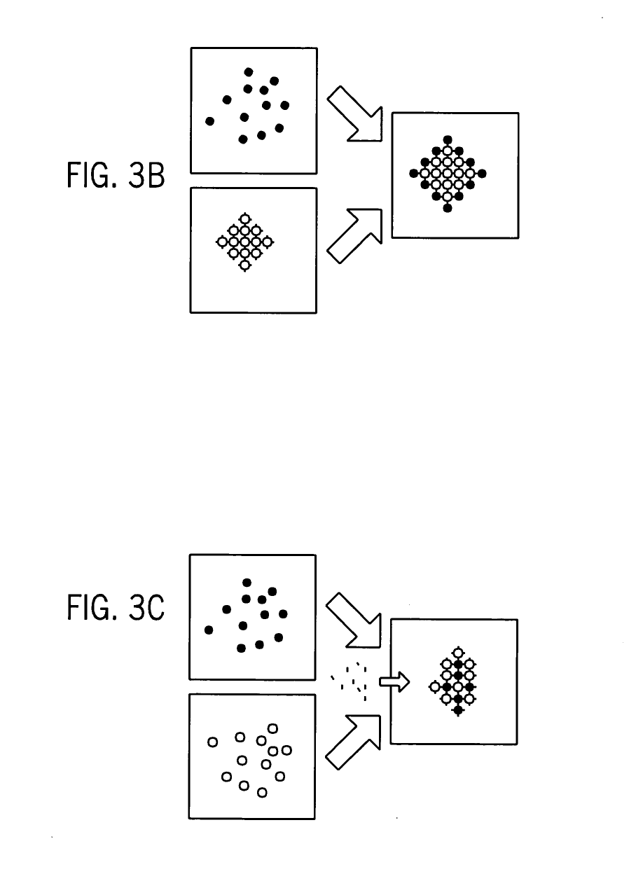

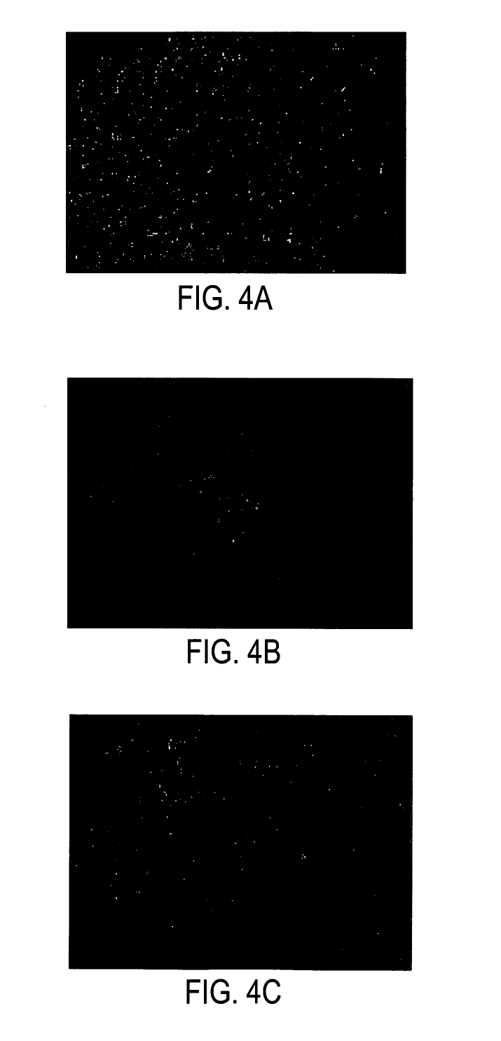

Mosaic or multi-species aggregates were also developed using the ConA lectin. Simple mosaics (FIG. 3) could be formed by mixing aggregates of one organism created under a lectin deficiency with those formed under excess (FIG. 3A). Size of the individual mosaics could be controlled by cell-lectin ratio. Alternately, free bacteria could be added to aggregates formed under a lectin-excess in order to create a lamellar structure (FIG. 3B). In such a fashion it is feasible to generate multi-lamellar aggregates with precision. Unordered aggregates (FIG. 3C) could be constructed by adding lectin to a mixture of free-swimming bacteria. These structures could be confirmed microscopically when one of the bacteria was bioengineered to produce green fluorescent protein. Eipfluorescence microscopy then reveals one organism as fluorescent green with the remainder non-fluorescent (FIG. 4).

D. Antimicrobial Susceptibility of Bacteria in Mono-Species Aggregates

Single species (Pseudomonas sp. 2881, Aeromonas hydrophila and Corynebacterium aquaticum) aggregates of 10, 50 and 100 μm in size were constructed and treated with different concentrations of three simple quaternary ammonium compounds (n-alkyl dimethyl benzyl ammonium chloride where n=12, 14 or 16). Results generated showed:

Aggregation, to any extent, of any of the three species resulted in a reduced susceptibility to each of the QUATs (Table 4).

The larger the size of any given aggregate the greater the reduction in the susceptibility (Table 4 and FIG. 5).

The survival curves were biphasic, with the second phase indicating a cessation of kill. The size of the fraction of surviving bacteria (at cessation/equilibrium) was dependent upon the amount of QUAT added, the susceptibility of the species (Table 5) in the single species aggregate to QUAT, the type of QUAT used (C12, C14 or C16), and the aggregate size (Table 4). Such tailing of survival curves is usually taken to indicate either a consumption of the available biocide (quenching) or the presence of a resistant sub-set of cells.

TABLE 4
Table showing the influence of single species
aggregate size and species used on total numbers of
survivors recovered after 1 mM C14 QUAT treatment.

| | Single-species aggregate | No. of survivors (%) |
|---|---|---|
| Strain | size (Diameter) | at 20 min |
| Pseudomonas sp. | No aggregate (free cells) | 0.001 |
| 2881 | Approx. 10 μm | 0.021 |
| | Approx. 50 μm | 0.0598 |
| | Approx. 100 μm | 0.5942 |
| Aeromonas | No aggregate (free cells) | <0.00001 |
| hydrophila | Approx. 10 μm | 0.0041 |
| | Approx. 50 μm | 0.0282 |
| | Approx. 100 μm | 0.4197 |
| Corynebacterium | No aggregate (free cells) | <0.00001 |
| aquaticum | Approx. 10 μm | 0.0002 |
| | Approx. 50 μm | 0.0027 |
| | Approx. 100 μm | 0.0829 |

TABLE 5
Table showing the influence of 1 mM QUATs of
different chain lengths (C12, C14 and C14) on 100 μm
single species aggregates on total numbers of survivors
recovered after a 20 minute treatment.

| | No. of survivors after 20 minutes following | | |
|---|---|---|---|
| | treatment of 100 μm aggregate with 1 mM QUAT | | |
| Aggregated strain | C12 QUAT | C14 QUAT | C16 QUAT |
| Pseudomonas sp. 2881 | 0.711 | 0.594 | 0.724 |
| A hydrophila | 0.635 | 0.420 | 0.594 |
| C. aquaticum | 0.240 | 0.083 | 0.266 |

Various explanations of the enhanced survival of bacteria in aggregates have been proposed in the past. These relate to:

i. The existence of physiological gradients across and established aggregate. In the current experiments the aggregation was complete within a few minutes. The physiology of the enveloped cells would therefore be relatively unchanged from that of planktonics.

ii. Reaction diffusion limitation restricting the access of antimicrobial to the core of the aggregate. This is refuted since the matrix polymers possess a diffusivity close to that of water, and the bulk phase would have to be depleted of biocide in order for protection to be permanently afforded to the deep lying cells. In the current experiments the lectins do not associate with the QUAT biocides but the aggregates provide a tortuous path to diffusion equilibration. Excess biocide remains in the bulk phase when the killing has reached equilibrium.

iii. The construction of the biofilm can only delay the achievement of diffusional equilibrium not prevent it. It is conceivable that a gradual exposure of bacterial cells to QUATS is less lethal to cells than a sudden exposure to full treatment levels (i.e. gradual increases in membrane surface pressure caused by insertion of a QUAT biocide can be accommodated because of transition of QUAT from outer leaflet to inner leaflet, whereas sudden exposure gives an asymmetric effect to the membrane resulting in rapid death and lysis). Thus a retardation of access might protect the deeper lying cells.

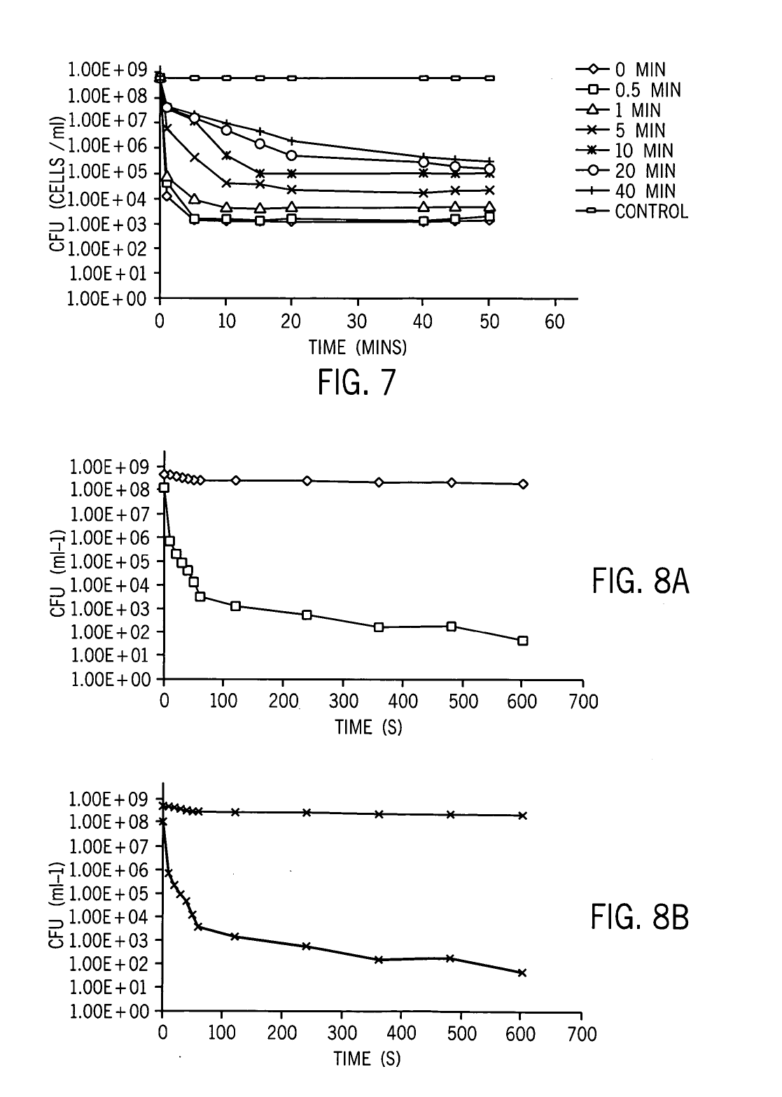

If (iii) were correct then the aggregate effect could be duplicated in planktonic systems by delivering the biocide in a controlled fashion to mimic the retardation found in aggregates. A syringe driver (FIG. 6) was used to gradually deliver QUAT to free floating cells. All three QUATs and all three species of bacteria were deployed in these experiments. Equivalent doses of QUAT were delivered as a bolus or over periods of time from 30 seconds to 50 minutes to a fixed density of cells. Results showed that as the rate of delivery of QUAT was decreased, the number of bacteria that survived (Plateau level) increased (Example data in FIG. 7). This confirmed that the protective effect of the aggregation was to retard biocide access rather than prevent it. Results also indicated that if formulations were engineered with permeabilisers, then provided equilibration of biocide could be achieved across an aggregate within 30 seconds, then the biofilm effect could be circumvented and the cells would be of a similar susceptibility to that of planktonic cells. Lectin-constructs therefore provide a convenient tool by which such formulations can be quantified and bench-marked.

E. Antimicrobial Susceptibility of Bacteria in Structured Multi-Species Aggregates

Binary lamellar aggregates were constructed for all combinations of the three test species. Core aggregate size was 50 microns diameter. Cores were coated with an equal number of the partner species. Each test species served as both core-aggregate and shell in these experiments. When free suspensions of these organisms were mixed and exposed to QUATS, there was no aggregation and no change in the survival pattern of either species relative to exposure in monoculture, indeed the data were super-imposable (Example data shown in FIG. 8A and 8B). When the cells were aggregated prior to exposure to QUATs, then it became apparent that there was not only a protection afforded by aggregation but that the relative location of one species to the other affected the inactivation. Selected data is presented that compares inactivation of unordered (homogeneous) aggregates of two different species with ones where each of the partner organisms serves as either the core or shell to the other. These effects were noted for all organism combinations and biocides tested.

Results indicated that:

The species that acted as the “shell” conferred protection to the species in core, regardless of its susceptibility to QUAT.

The greater protection was afforded by the most susceptible species; generally these had the greatest binding affinity to the QUAT (Tables 6, 7 and 8).

Retardation of biocide access, and hence the likely outcome of treatments within the core, is greatly affected by the spatial arrangement of clonal mosaics within biofilms.

TABLE 6
Sample data showing the influence of 1 mM C14
QUAT on Pseudomonas sp. 2881 - A. hydrophila
multi-species aggregates of 100 μm in size.

| | % No. survivors after 20 | |
|---|---|---|
| | minutes | |
| Pseudomonas sp. 2881 - A. hydrophila | Pseudomonas | |
| multi-species aggregate | sp. 2881 | A. hydrophila |
| Unordered mixture | 0.241 | 0.112 |
| Pseudomonas sp. 2881 shell, | 0.147 | 0.176 |
| A. hydrophila core | | |
| A. hydrophila shell, Pseudomonas sp. | 0.319 | 0.0065 |
| 2881 core | | |

TABLE 7
Sample data showing the influence of 1 mM C14
QUAT on Pseudomonas sp. 2881 - C. aquaticum
multi-species aggregates of 100 μm in size.

| | % No. survivors after 20 | |
|---|---|---|
| | minutes | |
| Pseudomonas sp. 2881 - A. hydrophila | Pseudomonas | |
| multi-species aggregate | sp. 2881 | C. aquaticum |
| Unordered mixture | 0.264 | 0.0412 |
| Pseudomonas sp. 2881 shell, | 0.219 | 0.0698 |
| C. aquaticum core | | |
| C. aquaticum shell, Pseudomonas sp. | 0.425 | >0.001 |
| 2881 core | | |

TABLE 8
Sample data showing the influence of 1 mM C14
QUAT on A. hydrophila - C. aquaticum
multi-species aggregates of 100 μm in size.

| | % No. survivors after 20 | |
|---|---|---|
| Pseudomonas sp. 2881 - A. hydrophila | minutes | |
| multi-species aggregate | A. hydrophila | C. aquaticum |
| Unordered mixture | 0.172 | 0.0364 |
| A. hydrophila shell, C. aquaticum core | 0.131 | 0.0457 |
| C. aquaticum shell, A. hydrophila core | 0.365 | >0.001 |

We claim:

1. A method of creating a bacterial aggregate comprising the step of:
combining planktonic bacterial cells with an effective amount of lectin, wherein the amount of lectin is effective to bind the bacterial cells together in an aggregate.

2. An aggregate created by the method of claim 1.

3. The method of claim 1 wherein the lectin is Concanavalin A.

4. The method of claim 1 wherein the bacterial cells are homogeneous.

5. The method of claim 1 wherein the bacterial cells are heterogenous.

6. The method of claim 1 additionally comprising the step of coating the bacterial aggregate with a second mixture of bacteria and lectin, whereby a lamellar aggregate is constructed.

7. The aggregate created by the method of claim 6.

8. A method of evaluating the efficacy of a biocide comprising the step of exposing the bacterial aggregate of claim 2 to the biocide and evaluating the viability of the bacterial cells within the aggregate.

9. A method of evaluating the efficacy of a biocide comprising the step of exposing the bacterial aggregate of claim 7 to the biocide and evaluating the viability of the bacterial cells within the aggregate.

10. A method of creating a microbial aggregate comprising the step of:
combining microbes with an effective amount of lectin, wherein the amount of lectin is effective to bind the microbes together in an aggregate.

11. The method of claim 10 wherein the microbes comprise at least one member from the group consisting of bacteria, yeast and fungi.

12. An aggregate created by the method of claim 10.

13. A method of evaluating the efficacy of a biocide comprising the step of exposing the aggregate of claim 12 to the biocide and evaluating the viability of organisms within the aggregate.
